(19) United States
(12) Patent Application Publication
(10) Pub. No.: US 2026/0033775 A1
(43) Pub. Date: Feb. 5, 2026

(54) RAPID EYE MOVEMENT SLEEP DISORDER DETECTION TO FACILITATE SELECTIVE SCREENING FOR PARKINSON'S DISORDER

(71) Applicant: NEUROVIGIL, INC., Moffett Field, CA (US)

(72) Inventor: Philip LOW, Moffett Field, CA (US)

(73) Assignee: NEUROVIGIL, INC., Moffett Field, CA (US)

(21) Appl. No.: 19/356,945

(22) Filed: Oct. 13, 2025

Related U.S. Application Data
(63) Continuation of application No. PCT/US2024/031494, filed on May 29, 2024.
(60) Provisional application No. 63/505,339, filed on May 31, 2023.

(51) Int. Cl. A61B 5/00; A61B 5/291; A61B 5/374

(52) U.S. Cl. CPC A61B 5/4812; A61B 5/291; A61B 5/374; A61B 5/4809; A61B 5/7264

(57) ABSTRACT

Detecting rapid eye movement (REM) sleep and associated abnormalities can be significant for identifying various neurological disorders. The present disclosure relates to detection of time intervals during which a subject is in REM sleep by leveraging encephalography (EEG) data from selective spectral frequencies. The techniques, as disclosed herein, may use one or more EEG electrodes, more specifically, a single-channel EEG signal offering a simple and a cost effective solution. The disclosed technique may preprocess this EEG data, extract features and/or derived features for one or more time intervals from selective spectral bands e.g., Delta and Gamma. These extracted features may be normalized and clustered to further determine REM, non-REM, and awake time intervals. By leveraging one or more non-EEG sensors to detect presence of muscle tones for the identified REM sleep intervals and by performing sleep pattern analysis, potential REM intervals may be validated for detection of REM behavior disorder.

### CROSS-REFERENCE TO RELATED APPLICATIONS

This application is a continuation of International Patent Application No. PCT/US2024/031494, filed on May 29, 2024, which claims the priority to and the benefit of U.S. Provisional Application No. 63/505,339, filed on May 31, 2023, which is hereby incorporated by reference in its entirety for all purposes.

### BACKGROUND

Rapid eye movement (REM) sleep behavior disorder (RBD) is a sleep disorder that may be characterized by absence of muscle paralysis during REM sleep, leading to enactment of dream content through vocalization or physical movements. RBD can be identified as a potential prodromal symptom of Parkinson's disease (PD) and other neurodegenerative disorders e.g., multiple system atrophy (MSA), and Lewy Body Dementia (LBD). Parkinson's disease is a progressive neurological disorder primarily associated with motor symptoms such as tremors, rigidity, and bradykinesia. However, non-motor symptoms, including sleep disturbances, often precede the onset of motor symptoms by a significant amount of time (e.g., spanning years). Emerging observations suggest a potential link between RBD and the development of Parkinson's disease. This association highlights that early detection and intervention for RBD could serve as a proactive strategy for identifying individuals at high risk of developing neurodegenerative disorders such as Parkinson's disease thereby facilitating timely clinical management.

Conventional methods for diagnosing RBD may rely on clinical assessments, questionnaires, and polysomnography (PSG), which involves monitoring of physiological signals including electroencephalography (EEG). For example, EEG electrodes can capture EEG signals that include neural signals, which can then be processed to detect different types of brain waves that are indicative of different non-REM stages of sleep. The conventional methods involving EEG alone may not be sufficient to detect RBD because during REM sleep, brain activity pattern can resemble those observed during wakefulness, making it difficult to differentiate between the two states. Additionally, RBD episodes may occur during REM sleep but may not always exhibit distinct EEG abnormalities, further complicating detection through EEG analysis solely. Incorporating reason for speech and the movements of a subject (e.g., capturing visual clues and behavior through video recordings, audios, or speech analysis) can provide additional context and insights, specifically for sleep disorders such as RBD.

However, incorporating video and/or audio data into sleep monitoring can be costly, time-consuming, and inconvenient, posing barriers to accessibility for subjects. The procurement and maintenance of specialized equipment, along with a technical expertise in data acquisition and analysis may contribute to overall expense and complexity in implementing comprehensive sleep monitoring solutions. Additionally, accessibility to sleep monitoring facilities equipped with these advanced technologies may be limited in certain geographic areas or healthcare settings, leading to disparities in healthcare access and diagnostic capabilities. Addressing these challenges may require the development of more affordable and widely accessible sleep monitoring technologies that can provide reliable insights into sleep disorders while minimizing burden on healthcare facilities.

### SUMMARY

Certain aspects and the features of the present disclosure relate to identification of various sleep stages including rapid eye movement (REM) sleep via one or more electroencephalography (EEG) signals. The present disclosure may further determine REM behavior disorder (RBD) by leveraging one or more non-EEG sensors to detect muscle tones for the identified REM sleep and by performing a sleep analysis. One or more first signals from one or more EEG electrodes (e.g., at least one active electrode, a reference electrode, and potentially a ground electrode) may be received over a period of time. At least one of these EEG electrodes may be positioned on a subject during the period of time. In a preferred embodiment, the one or more first signals include a single-channel EEG data. One or more second signals from non-EEG electrodes (e.g., electromyography (EMG) sensor or accelerometer) may also be received, where at least one of these non-EEG electrodes may be positioned on, near or in the subject during the period of time. The disclosure may further preprocess these one or more first signals from EEG data (e.g., removing artifacts manually or automatically or a combination thereof, normalizing one or more time intervals for differences in power across time to determine appropriate frequency bands suitable for feature extraction). The features and/or derived features may be extracted for the one or more time intervals from selective spectral bands e.g., Delta and Gamma. These extracted features may be further normalized (e.g., z-scoring or quantile transformation for making different features or derived features to be on same scale) and clustered to determine time intervals that correspond to REM, awake and non-REM (e.g., any one of the sleep Stages 1 to 4). REM sleep may be characterized by vivid dreaming and high brain activity resembling wakefulness. Therefore, the predicted subset of multiple time intervals that correspond to potential REM time intervals may be further validated for detection of REM behavior disorder (RBD) by leveraging one or more non-EEG sensors to detect muscle tones i.e., detecting whether the muscles are relaxed depicting normal REM sleep having muscle atonia or presence of a muscle tone (tension in a muscle showing readiness for action or a posture) in the subject during the REM time windows.

Additionally, a sleep analysis may be performed, via one or more modeling techniques, to validate the subset of the multiple time intervals corresponding to REM stage of sleep. The one or more modeling techniques may be trained to learn a temporal structure comprising different stages of sleep within a given period of time. In some aspects, the one or more modeling techniques may be configured to identify a smooth transition between different stages of sleep by analyzing neighboring time intervals for each time interval of the subset of the multiple time intervals corresponding to REM stages of sleep. For example, if a specific time interval from the subset of multiple time intervals is surrounded by “Stage 2” windows, the one or more modeling techniques may be configured to predict that the specific window (or time interval) corresponds to REM sleep. Moreover, the one or more modeling techniques may also be configured to identify, within the period of time, a gradual increase of a consecutive REM stages of sleep from the subset of the multiple time intervals corresponding to REM stage of sleep.

Based on the prediction from the non-EEG sensors depicting presence of a muscle tone during the predicted subset of multiple time intervals that correspond to REM state and sleep analysis performed by the one or more modeling technique, it may be predicted whether the subject is diagnosed with RBD. In response to this prediction, an output indicating the prediction results of RBD may be shown. If, for example, a subject is diagnosed with RBD, one or more actions can be triggered. These actions may include, but not limited to, generating alerts, notifying the concerned authorities (e.g., medical staff, relatives and/or the subject) for a complete medical evaluation for confirming the diagnosis for a neurodegenerative disease such as Parkinson's, scheduling a consultation with a sleep specialist for thorough neurological examination.

EEG signals may be characterized by different frequency bands, each associated with specific cognitive and physiological states. For example, Stage 3 of non-REM sleep may be associated with Delta band that is characterized by slow waves (or frequencies) with high amplitudes. Similarly, Gamma may be characterized by very high frequencies of EEG signals with low amplitudes, associated with high-level information processing and perception such as REM sleep. The extracted set of features and/or derived features associated with these one or more frequency bands may include e.g., Delta power, Gamma power, standard deviation, maximum amplitude, Gamma power/Delta power, time derivative of Delta, and time derivative of Gamma power/Delta power.

The one or more modeling techniques can model sequential data and learn the probability of transitions between sleep stages. In some instances, the one or more modeling techniques include Hidden Markov Model (HMM) and/or recurrent neural network (RNN).

In some instances, the one or more first signals from EEG electrodes and the one or more second signals from non-EEG sensors that may be received over a period of time, may be segmented into multiple time intervals during, or prior to, the preprocessing.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an exemplary block diagram that performs feature extraction for determining one or more sleep stages of a subject from one or more encephalography (EEG) channels.

FIG. 2 shows an exemplary overview for determining the one or more sleep stages of the subject from the one or more EEG channels.

FIG. 3 illustrates example EEG waveforms of the subject recorded for rapid eye movement (REM) state and awake state for different time windows.

FIG. 4 illustrates an example overview for determining REM behavior disorder (RBD) by leveraging one or more non-EEG sensors.

FIG. 5 illustrates an example depiction of a normal sleep cycle for the subject.

FIG. 6 illustrates an example architecture for predicting RBD in accordance with some aspects of the present disclosure.

FIG. 7 illustrates an example process flow for determining RBD by leveraging the one or more EEG channels, additional non-EEG sensors and sleep analysis.

FIG. 8 is an example illustration of a computer system in which various embodiments of the present disclosure may be implemented.

### DETAILED DESCRIPTION

Detecting rapid eye movement (REM) sleep and associated abnormalities can be significant for identifying various neurological and psychiatric disorders. For example, Narcolepsy characterized by abnormal REM patterns, REM behavior disorder (RBD) characterized by loss of muscle atonia during REM sleep, Parkinsons's disease characterized by RBD, Lewy Body Dementia (LBD) associated with RBD, Alzheimer's disease associated with non-REM sleep disturbances, depression, and mood disorders. The present disclosure relates to detection of particular time intervals during which a subject is in REM sleep by leveraging encephalography (EEG) data from selective spectral frequencies. The disclosed technique may preprocess this EEG data, extract features and/or derived features for one or more time intervals from selective spectral bands e.g., Delta and Gamma. These extracted features may be further normalized to be drawn on a same scale and clustered to further determine various stages of sleep including REM, non-REM and awake time intervals. By leveraging one or more non-EEG sensors that detect presence of muscle tones in the subject for the identified REM sleep intervals and by performing sleep analysis, potential REM intervals may be further validated for the detection of REM behavior disorder (RBD).

The techniques, as disclosed herein, may use one or more EEG electrodes, more specifically, a single-channel EEG signal (i.e., at least one active electrode, a reference electrode, and potentially a ground electrode) offering a simple and a cost effective solution. Using a single-channel EEG signal may be challenging for detecting various stages of sleep (including REM) as compared to conventional multi-channel EEG setups. This is because the single-channel EEG signal may have a limited spatial resolution, limited coverage of the EEG signals as sleep stages may be characterized by complex and distributed patterns of neural activities across different brain regions, lack of generalization ability i.e., variability of sleep patterns (and resulting EEG signals) among individuals.

In some embodiments, the present technique can capture brain activities to determine various time intervals indicative of REM sleep by strategically placing the pair of EEG electrodes on scalp. When a subject (e.g., an animal or a human) falls asleep, sleep lightens into intermediate sleep stages and can enter a sleep state known as REM characterized by high frequency low-power EEG activity. EEG signals may follow a 1/f distribution i.e., the higher frequency signals in EEG tend to have smaller amplitudes and therefore lower spectral power and vice versa. This EEG data may be pretreated (or preprocessed) for artifacts that may occur due to e.g., muscle activities, movements, or high-frequency noise. These artifacts may be removed manually or automatically with various techniques including filtering, independent component analysis (ICA), principal component analysis (PCA) or template matching. EEG signals are typically examined in time intervals of similar or varying lengths, i.e., when the EEG signal is used for analyzing sleep, it may be segmented (after, or prior to artifact removal) into multiple time intervals (or windows) where each time interval represents a portion of data from the EEG series. For further analysis, scanning windows and sliding windows can be used to separate the EEG data into time series increments.

For each time window, a differential EEG signal may be normalized for one or more time windows for differences in power across time to determine appropriate frequency band suitable feature extraction. Such normalization can reveal low power, statistically significant shifts in power at various frequency bands (e.g., Delta and Gamma). It should be understood that any frequency band (or range) may be utilized. In some instances, Gamma and Delta bands are utilized for extracting features e.g., Delta power, Gamma power, standard deviation, maximum amplitude and/or derived features such as Gamma power/Delta power, time derivative of Delta, time derivative of Gamma power/Delta power and the like as robust features to extract REM intervals. These derived features may be clustered to group similar time intervals of sleep based on the common spectral characteristics. To perform clustering effectively, the features (or derived features) including e.g., two or more of these features may be normalized again to be on a comparable scale. Subsequent to clustering where each cluster may represent a set of similar spectral characteristics, sleep stages can be assigned to multiple time intervals based on (pre-defined or dynamic) thresholding. By analyzing the distribution of normalized derived features within each cluster, thresholds may be defined to separate different clusters, for example, If the power exceeds a particular threshold, the window may be assigned as a “potential REM” window. Other time windows may be assigned to a non-REM state i.e., “Stage 1”, “Stage 2”, “Stage 3”, or “Stage 4” or an awake state.

Further information about processing of EEG data to facilitate REM detection can be found at U.S. application Ser. No. 13/129,185, filed on Nov. 16, 2009, which is hereby incorporated by reference in its entirety for all purposes.

In one aspect of the present disclosure, one or more non-EEG sensors may be used to further validate potential REM sleep intervals thus avoiding false positives. The incorporation of such multi-modal data i.e., non-EEG sensors (e.g., electromyography (EMG) and/or accelerometer) individually, or in combination may provide an improved assessment of REM sleep detection leveraging distinct physiological features associated with this sleep stage. During normal REM sleep, there is a natural reduction in muscle tone-a state known as atonia in which muscles are relaxed showing minimal electrical activity when detected by a sensor e.g., an EMG sensor. These muscle tones may represent a baseline level of tension or stiffness in the muscle at rest that helps in maintaining posture, joint stability and readiness for action, thus making it easier for a limb or body to quickly move. In RBD, atonia is absent partially or completely leading to an increased muscle tone or even show body movements instead of showing a relaxed muscle state. The one or more non-EEG sensors may alternatively or additionally be attached to or integrated within a wearable device (e.g., a cap or headband). In some instances, a single device (e.g., a patch with adhesive, a cap, a headband, etc.) may be configured to include or to receive (e.g., mechanically connect with) the one or more non-EEG sensors. In some instances, one or more EMG sensors are used to detect higher than normal muscle tones that may depict a continuous and passive contraction of muscles during REM sleep. By measuring muscle tones with one or more EMG sensors during REM sleep may be an indicator for synucleinpathy or its early stages.

In some other instances, one or more non-EEG sensors include accelerometers that may be used to capture body movements during REM sleep. The data from these one or more non-EEG sensors may be extracted for the same time intervals as that of potential REM time intervals. This extracted (or segmented) data may be preprocessed i.e., normalized (for consistency and reducing noise) and performing feature extraction e.g., calculating power, RMS value or entropy. The data (preprocessed, partially preprocessed or raw) from one or more non-EEG sensors, e.g., EMG sensors and/or accelerometers may be combined with potential REM time intervals. The data from these sensors may be fed into one or more prediction models such as a machine-learning models configured to detect abnormal body movements (i.e., kicking, punching, flailing, talking or shouting, thrashing, leaping out of bed etc.) and/or abnormal muscle tones during REM sleep (i.e., higher tension in muscles as opposed to normal REM sleep that cause muscles to be relaxed due to muscle atonia) leading to reliable detection of RBD. For example, sensor data from one or more accelerometers and one or more EMG sensors may be aggregated within each modality and fed into separate prediction models that are trained to detect corresponding movements.

During REM sleep, using non-EEG sensors (e.g., an accelerometer) may trigger false alarms due to unrelated movements such as those from a bed partner or muscle twitches caused by external factors. EMG sensors are generally mounted on muscles for capturing muscle tone, therefore, the movements from the partner may be unlikely to produce significant tension in the muscles. Moreover, to distinguish true signals from noise, sensitivity thresholds can be adjusted to filter out insignificant muscle tension or movements. Additionally, sensor fusion, i.e., combining EMG and accelerometer (or electrocardiogramay provide cross-validation, potentially reducing false alarms by cross-referencing data from different sensors.

Performing an analysis of sleep cycles that may involve identifying abnormalities during REM sleep and specific patterns that are indicative of disorder may also contribute to detection of RBD. For example, given a long sleep time e.g., a night sleep of approximately 8 hours, REM windows tend to lengthen and occur more frequently in the latter half of the total sleep. During the first half of the sleep, more time may be spent in Stage 3 or Stage 4 (deep sleep) and less time in REM sleep. Additionally, given that humans and animals generally transition in a smooth way between various sleep stages, it may be predicted—for each “potential REM” window—whether the window corresponds to when the subject was awake or was in REM sleep. For example, if a “potential REM” window (or a set of consecutive “potential REM” windows) is surrounded by “Stage 2” windows, one or more modeling techniques may be configured to predict that the window (or set of consecutive windows) corresponds to REM sleep. Meanwhile, if a “potential REM” window (or set of consecutive “potential REM” windows) is surrounded by “Stage 1” windows, the one or more modeling techniques may be configured to predict that the window corresponds to an awake state.

To perform sleep analysis, one or more modeling techniques may be trained to learn a sequential structure comprising various sleep stages throughout a complete sleep. Modeling techniques such as recurrent neural network (RNN), long short-term memory network (LSTM), gated recurrent unit (GRU) or Hidden Markov Model (HMM) may be used for sleep pattern analysis and temporal analysis. These techniques can model sequential data thereby capturing the temporal context by automatically estimating the relationship between consecutive time intervals. Such modeling techniques can learn temporal structure and consider the probability of transitions between sleep stages. By training one or more models on labeled sleep data segmented in aggregating time intervals or overlapping windows of time intervals (that may or may not be including EEG data and/or data from one or more non-EEG sensors), statistical relationships and probabilities of transitioning between different sleep states based on the observed sequence can be learnt and use this information to correct inconsistencies. It should be understood that to perform various aspects of sleep analysis, one unified model or different models may be trained. Once a model is trained, it may be applied to a new input segmented sleep data to validate sequence of sleep stages. The trained model can look for abnormal patterns and transitions of sleep stages. In RBD, the transitions into or out of REM may occur more frequently or in unusual manner such as frequent interruptions of REM by wakefulness or other stages. Such networks may be trained to validate each interval labeled as potential REM, awake or non-REM states based on the observed patterns.

The prediction results from the one or more modeling technique that may perform sleep analysis and the prediction model that may detect muscle tone during REM window may be combined to obtain a final prediction. This combination may be performed by various techniques including simple average, weighted average, majority voting or other similar techniques. On identification of RBD based on the predictions, one or more actions can be performed. Such predictions may be incorporated into a system that can perform one or more actions related to both clinical interventions and safety measures to improve health of the subject and reduce the risks of injury. These actions may include, but not limited to, generating alerts, notifying the concerned authorities (e.g., medical staff, relatives and/or the subject) for a complete medical evaluation for confirming the diagnosis for a neurodegenerative disease such as Parkinson's, scheduling a consultation with a sleep specialist for thorough neurological examination.

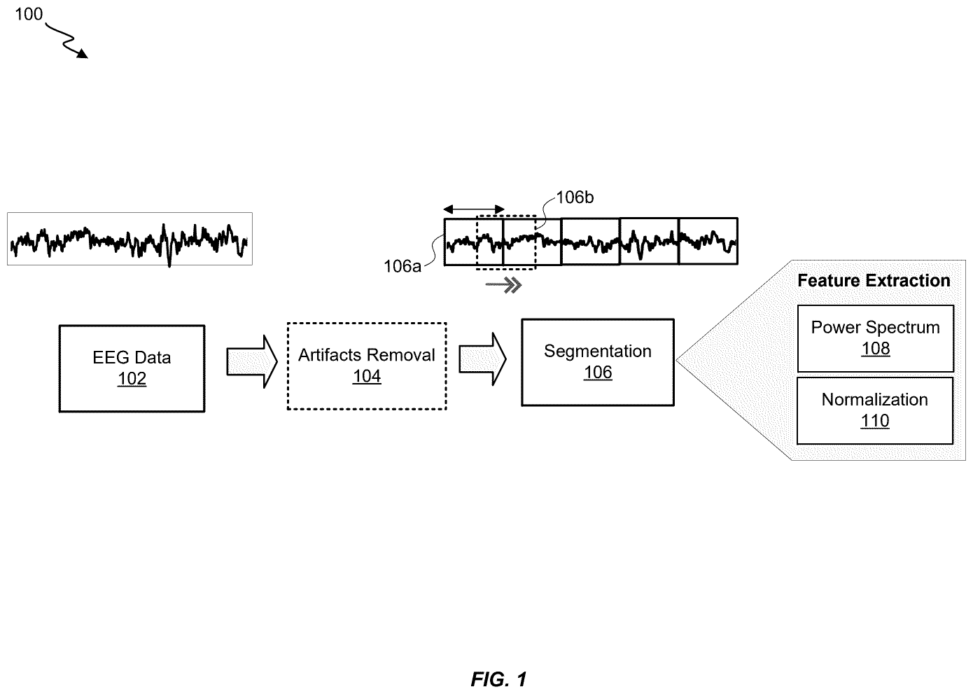

FIG. 1 illustrates an exemplary block diagram that performs feature extraction for determining sleep stages of a subject from one or more encephalography (EEG) channels. For example, electroencephalography (EEG) data 102 can be received from a single channel or multiple channels. This EEG data 102 may be optionally treated for removing artifacts, where an artifact refers to any part of EEG data that misrepresents the data intended to be received (e.g., movement data in an EEG signal). These artifacts may occur due to e.g., muscle activities such as jaw clenching or head movements causing high-frequency noise, periodic disturbances caused by electrical activity of heart, or other environmental artifacts such as electromagnetic interferences, thereby impacting the accuracy of sleep stage observation. These artifacts can be removed at 104 from the EEG data 102, for example, by manually removing i.e., by visually inspecting EEG signals (and/or in parallel observing the subject) and rejecting segments that include large-amplitude fluctuations or sudden changes that are likely to be artifacts or automatically filtering out of EEG data 102 during, prior to, or after segmentation 106 via a filtering (e.g., DC filtering) or data smoothing technique.

The EEG data (or signals) 102 can also be pretreated with component analysis i.e., by decomposing EEG signals into independent components, identifying and removing artifacts (i.e., at 104) based on the spatial and temporal characteristics. EEG artifacts may also be removed by estimating the artifact subspace using methods e.g., PCA and projecting the EEG data 102 onto orthogonal subspace for artifacts removal 104. In another instances, template matching may be performed that may identify and remove known artifact patterns by comparing the EEG data 102 with predefined templates. Additionally, wavelet transform may be applied that decomposes the EEG data 102 into different frequency components using wavelet transform and remove artifacts in specific frequency bands.

After (or prior to) treating the artifacts, EEG data 102 (or EEG signal) segmentation 106 may be performed that splits the EEG time series data 102 into multiple time intervals (of similar or varying lengths) via a variety of separating techniques, where each time interval is a portion of data from the EEG series. The time intervals may be segmented into different subsections using a scanning window 106a. For example, a one hour time frame of a received EEG signal can be scanned in increments of 1 minute (i.e., a scanning window of 1 minute), thus resulting in 60 discrete time segments. The scanning window 106a can use a sliding window 106b, where sections of the sliding window 106b may have overlapping time series sequences. For example, the one hour time frame of the received EEG signal can be scanned with a 1-minute scanning window that begins every 30 seconds (i.e., a sliding window of 30 seconds), thus resulting in a 1-minute scanning window that overlaps by 30 seconds. Scanning windows 106a and sliding windows 106b can be used to separate the EEG data 102 into time series increments. In one aspect, the EEG signal may be adjusted to account for differences in power by normalization 110. For this purpose, power spectrum 108 may be calculated e.g., by calculating power spectral density of each interval of the EEG data 102. The power may be calculated by different techniques such as multi-taper transform, Fournier transform, or wavelet transform and then any form of normalization 110 may be performed by weighing the spectral power of the one or more time intervals across time. The normalized power of each time interval at one or more frequencies across time may help determining appropriate frequency windows for extracting information. Such normalization 110 can reveal low power and statistically significant shifts in power at one or more frequencies (e.g., Delta band, Gamma band, and the like).

EEG signals may be characterized by different frequency bands, each associated with specific cognitive and physiological states. For example, Delta band that ranges typically around [0.5-4] Hz comprising slow waves or frequencies with high amplitudes. Deep sleep such as Stage 3 of non-REM sleep that supports restorative processes may be associated with this band. Similarly, Theta band that may range approximately around [4-8] Hz, comprises moderate frequencies and amplitude. Light sleep such as Stage 1 and 2 of non-REM sleep, drowsiness, meditation, or similar states may be associated with this frequency band. Alpha band may range approximately around [8-12] Hz and may characterize moderate frequencies with lower amplitudes than Delta and Theta band. Various states such as relaxing, wakefulness or closed eyes may be associated with Alpha band. Additionally, Alpha band may facilitate the transition between wakefulness and sleep. Followed by Alpha, Beta band approximately ranging from [12-30] Hz may be characterized by higher frequency with lower amplitude that may be associated with active thinking, focus, wakefulness, or similar activities. The frequency band with relatively higher frequencies, Gamma, approximately ranging [30-100] Hz may be characterized by very high frequencies of EEG signals with low amplitude. Gamma band may be associated with high-level information processing and perception such as REM sleep that may be characterized by vivid dreaming and high brain activity resembling wakefulness. By processing these spectral characteristics of spectral bands, various sleep stages may be assigned to segmented time intervals.

Among these frequencies, any frequency band can be revealed and utilized for analysis. Features can be calculated for each time interval after appropriate frequency windows have been established. Such features can include low frequency power (e.g., Delta power and Theta), high frequency power (e.g., Gamma power), standard deviation, maximum amplitude (e.g., maximum of the absolute value of peaks) and the like. Further calculations can be done on the calculated features for each time interval creating derived features such as Gamma power/Delta power, time derivative of Delta, time derivative of Gamma power/Delta power and the like. Time derivatives can be computed over preceding and successive time intervals. These derived features may be clustered to group similar time intervals of sleep based on these spectral characteristics or the normalized features. To perform clustering 202 effectively, the normalized features including e.g., two or more of these parameters may be preferred to be on a comparable scale. Therefore, normalization 110 can be performed again for the calculated information across the time intervals for enabling different derived features to be on same scale. A variety of data normalization 110 techniques can be conducted including z-scoring, min-max scaling, quantile transformation, log transformation and other similar techniques. In some instances, normalization 110 is performed by z-scoring that is a statistical technique to standardize the range of independent variables (or features). It may involve transforming the features such that the features have a mean of zero and a standard deviation of one. By applying z-scoring, different derived features of the spectral power data such as Delta power and Gamma power/Delta power may be scaled to a common range, thus eliminating biases.

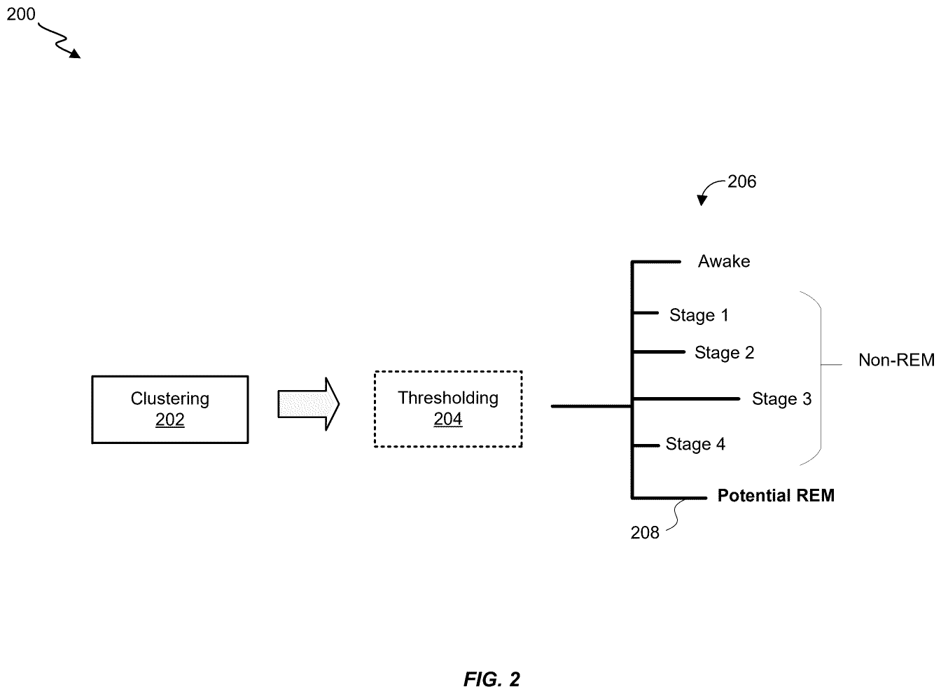

FIG. 2 shows an exemplary overview for determining sleep stages of the subject from the one or more EEG channels (or single-channel EEG signal) 102. In some instances, the normalized features (such as Delta Power, Gamma power, standard deviation, maximum amplitude of Gamma or Delta) can be input into a classification technique (e.g., supervised machine learning techniques including Decision Trees, support vector machine (SVM), Random Forest, or a customized neural network trained on labeled sleep data) or a clustering technique 202 e.g., k-means clustering, hierarchical clustering or Gaussian mixture model (GMM). Additionally, component analysis such as PCA or ICA can also be used to determine the parameter space (e.g., types of information used) in the clustering. Subsequent to clustering 202 where each cluster may represent a set of similar spectral characteristics and optional thresholding 204, sleep stages can be assigned to multiple time intervals. By analyzing the distribution of normalized derived features within each cluster, thresholds may be defined to separate different clusters. For example, if for a specific cluster, Alpha power is below a certain threshold and/or Beta power exceeds a certain threshold (predefined or dynamic e.g., by calculating mean and standard deviation), the cluster may be assigned “awake”. The labeled time intervals within each cluster can then be presented as representations of sleep states in the subject for those periods of time.

In some aspects of the present disclosure, the exemplary system 100 may analyze the (single-channel) EEG signal 102 within multiple time windows to extract features that may be processed further (by the exemplary system 200) to identify potential REM time intervals 208. The features may be extracted by differential transformations applied to the EEG signal (e.g., first or second time derivative for highlighting changes in EEG signal), followed by the detection of power in gamma band. If the power exceeds a specified threshold, the window may be categorized (as illustrated in 206) as a “REM” window. Other time windows may be considered “Awake” or non-REM windows assigned to various sleep stages such as “Stage 1”, “Stage 2”, “Stage 3”, and “Stage 4”.

Additionally, artifact information (e.g. movement data, poor signal data, or the like) can also be utilized in the classification or clustering 202. For detected REM sleep intervals, artifacts (e.g. movement data, poor signal data, and the like) can also be used in sleep state classification. For example, artifacts can be used to analyze whether time interval or windows initially assigned a sleep state designation should be reassigned a new sleep state due to neighboring artifact data. For example, a time window assigned a REM state that has a preceding movement artifact or awake window can be reassigned an awake state. Further, for example, an artifact window that has a succeeding Stage 3 (SWS) window can be reassigned a Stage 3 state because there is a high likelihood that the time window represents a large Stage 3 sleep window rather than a large movement artifact, which is more common during wakefulness. In such ways, for example, artifact data can be utilized in a data smoothing technique.

Assigning sleep stages from Stage 1 to Stage 4 may involve analyzing EEG signals that are recorded during sleep and identifying specific patterns associated with each sleep stage. Automated algorithms, such as the disclosed technique, can perform EEG waveform analysis based on waveform characteristics such as amplitude, frequency, duration of sleep stages throughout the recorded sleep. For sleep cycles, “Stage 1” sleep is the lightest non-REM sleep stage and may occur as a subject transition from wakefulness to deeper sleep stages. During Stage 1 sleep, EEG signals may exhibit theta waves, which are slower in frequency compared to the alpha waves observed during wakefulness. This stage may be characterized by drowsiness, muscle relaxation, and occasional muscle twitches. Followed by Stage 1, “Stage 2” sleep is a deeper stage of non-REM sleep and may constitute the majority of the sleep cycle of a healthy adult. It may be characterized by the presence of sleep spindles (i.e., short bursts of high-frequency brain activity) and K-complexes (i.e., large bursts of slow waves) in the EEG-signal. Stage 2 sleep may be associated with further relaxation of muscle tone and a decreased heart rate and body temperature. In some examples, Stage 1 and Stage 2 are associated with intermediate sleep (IS) as these stages represent lighter phases of non-REM sleep. The IS states tend to act as a transition state between REM and SWS. “Stage 3” sleep, also termed as slow-wave sleep (SWS) or deep-sleep, may be characterized by a high amplitude and low frequency EEG signals (i.e., presence of slow delta waves in the EEG signals). It may be considered as the deepest and restorative stage of sleep during which the body undergoes physiological repair and recovery. Therefore, Stage 3 sleep may impact the physical health, immune functioning, and cognitive functioning significantly. “Stage 4” sleep that may be characterized by the maximum proportion of Delta waves (e.g., more than 50%) in the EEG signal, is another term for deepest phase of SWS. Both stages, (i.e. Stage 3 and Stage 4) are generally grouped together as SWS based on similar characteristics. During Stage 4 sleep, the body may experience minimal physiological activity, and awakening thresholds may be highest.

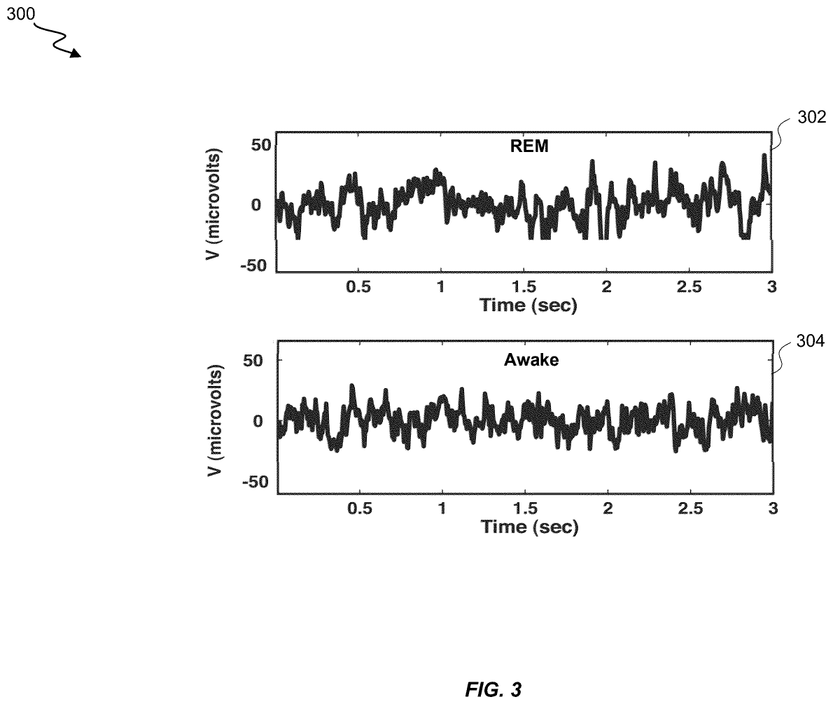

FIG. 3 illustrates examples of EEG waveforms of a subject recorded for REM state and awake state for different time windows. REM may be characterized by a very low amplitude “awake-like” EEG signal 302, (e.g., approximately ±30 μV) with higher power in Gamma than non-REM. The EEG waveform 302 during REM sleep can closely resemble awake EEG waveform 304 seen during wakefulness, hence the term “awake-like”, as illustrated in FIG. 3. During REM sleep, eyes start moving rapidly behind closed eyelids, and brain activity may increase, resembling patterns seen when the subject is awake. Additionally, as REM sleep may be characterized by e.g., vivid, complex, and often narrative-like dreams, the high level cognitive activity may become similar to the active thought processes and consciousness that is experienced during awake state. In some instances, this similarity can make it challenging to distinguish between REM sleep and wakefulness using EEG alone. Both REM sleep state and wakefulness may show resembling features such as low-amplitudes and high frequency EEG waves. Therefore, additional non-EEG sensors such as accelerometer or electromyography (EMG) sensors may be used to further enhance accuracy of the detected REM sleep intervals by avoiding false positives.

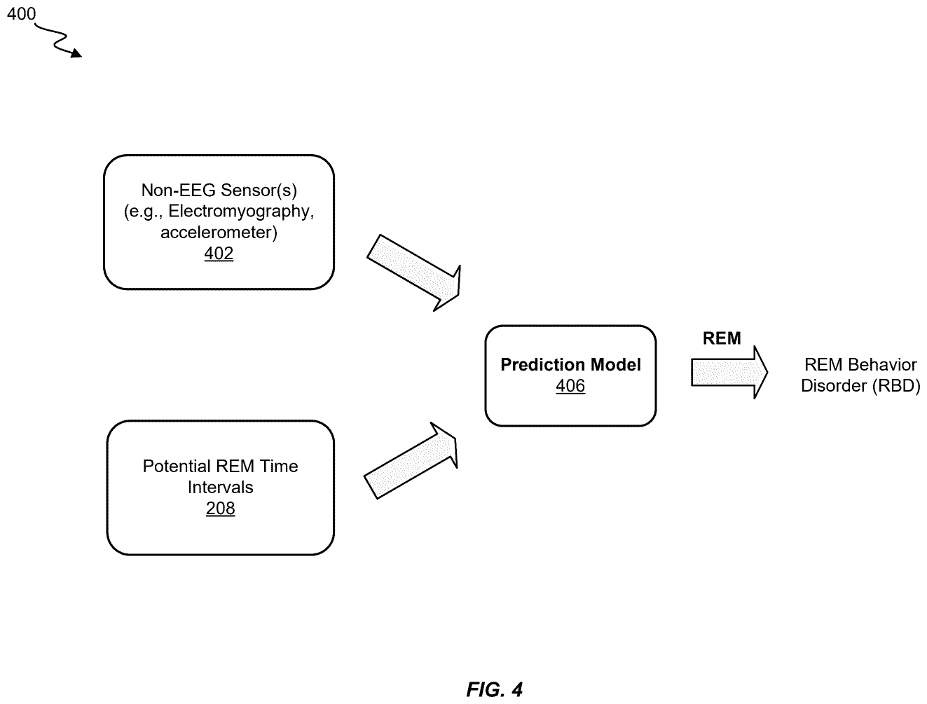

FIG. 4 illustrates an example block diagram 400 for detecting potential REM behavior disorder (RBD) from a subset of potential REM time intervals 208. REM sleep abnormalities may be a significant indicator of neurodegenerative disorders such as Parkinson's disease and synucleinopathies. One key indicator may be the RBD, where the usual muscle atonia (i.e., loss of muscle tone) during REM sleep occurs, leading to dream enactment behavior e.g., talking, shouting, impulsive or violent movements. These conditions may often precede the motor symptoms of Parkinson's disease by many years, thus contributing as early diagnostic sign. Other conditions may include changed REM sleep patterns such as shortened REM sleep portions, decreased REM latency (i.e., altered duration of entering first REM sleep period) autonomic dysregulation including irregular or abnormal heart rate and breathing patterns may also be considered as notable indicators. To monitor these indications, one or more non-EEG sensors 402 may be used to accurately assess REM sleep intervals and avoid false positives. Such non-EEG sensors may include, but not limited to, electromyography (EMG) sensors, electrooculography (EOG), accelerometer, heart rate monitors such as electrocardiogra (ECG) that may detect the increased heart rate variability during REM sleep as compared to non-REM sleep, respiration monitors for measuring irregular breathing patterns, and thermocouples that measures skin temperature changes.

In one instance, one or more EMG sensors can be used to detect muscle tone during REM sleep when it is expected to be minimum or absent (due to atonia)—an indicator for synucleinpathy or its early stages. Muscle atonia typically occurs during REM sleep, resulting in a low EMG signal activity (as opposed to signals representing brief twitches). While in wakefulness, muscle activity is comparatively higher and more continuous. Therefore, non-EEG sensors 402 such as EMG electrodes for capturing muscle tones, movements of muscles (or nerve signals being sent to a muscle) placed at e.g., chin, face, eyes, or neck may help to confirm the muscle atonia, which is a characteristic of REM sleep and distinguish it from muscle movement that is attributed to wakefulness. Even if a subject moves another part of the body, small muscle movements may still be detected (e.g., in the face). The one or more non-EEG sensors 402 may alternatively or additionally be attached to or integrated within a wearable device (e.g., a cap or headband). In some instances, a single device (e.g., a patch with adhesive, a cap, a headband, etc.) may be configured to include or to receive (e.g., mechanically connect with) the one or more non-EEG sensors. Alternatively, or additionally, one or more EOG sensors may be used to determine eye movement during REM sleep because rapid and irregular movement of eyes may be strongly attributed to REM sleep. Compared to REM sleep, wakefulness may involve more controlled and purposeful eye movements. For the detection of REM, EOG sensors may be placed near the eyes further confirming the accuracy of detected REM sleep intervals.

In some instances, non-EEG sensors 402 may include one or more accelerometers, which may be placed and/or configured to detect body movements. The non-EEG sensors (such as an accelerometer) can be non-invasive devices and receive measurements along multiple axes (e.g., x, y, and z) to measure the acceleration of body movements across these axes. When leveraged during REM sleep monitoring, accelerometers can provide data on frequency of movements, intensity of movements, duration of movements and other similar attributes that may help in identifying abnormal motor activities during REM sleep. For example, an increased frequency of high-intensity movements during REM sleep intervals may be flagged for the diagnosis of potential RBD in the subject. As REM sleep is attributed to minimal body movements due to muscle atonia (except for minor twitches), accelerometer data may be used to differentiate relative stillness attributed to normal REM sleep as opposed to body movements that are typically attributed (for healthy patients) to wakefulness. One or more accelerometers can be placed or attached to various parts of the body such as wrist, ankles, and torso, to capture body movements during REM sleep indicating RBD. The data collected by accelerometers can be segmented and analyzed for the same time intervals as predicted for REM sleep to analyze motion patterns. The incorporation of such multi-modal data (that includes signals from EEG sensors and one or more, non-EEG sensors 402 individually, or in combination) may provide an improved assessment of REM sleep detection leveraging distinct physiological features associated with this sleep stage.

It may be difficult to predict whether a person is exhibiting movement inconsistent with REM sleep using one or more non-EEG sensors 402. For example, a type of non-EEG sensor 402 may be configured to detect motion signals that may reflect movements that are not actually related to the subject being monitored. For example, movements from a bed partner or spontaneous muscle twitches or jerks that may occur due to external causes. These movements may be picked up by these non-EEG sensors and may have the potential to be misinterpreted as the movements from the monitored subject, thus leading to inaccurate or erroneous results.

In some instances, EMG sensors can be placed directly on the muscles of the monitored subject, such that the movements from the bed partner may be unlikely to produce significant muscle activities in the subject. Moreover, various signal processing techniques or filtering techniques may be used to identify true signal from such noisy signals. By adjusting the sensitivity thresholds, these sensors can be tuned to ignore minor or irrelevant movements. Sensor fusion may also be leveraged to reduce false alarms, i.e., by combining data from different modalities (or sensors) it may be possible to cross-validate. For example, using EMG and accelerometer, if both sensors pick movements simultaneously that may indicate a true signal.

To validate a subset of potential REM time intervals 208 for further determining RBD, one or more prediction models 406 may be used that takes the data from one or more non-EEG sensors 402. The EMG sensors may be configured and/or positioned to capture muscle activity, which may provide informative signals to distinguish between REM and non-REM sleep stages (or wakefulness). Other non-EEG sensors (such as accelerometers for capturing body movements, EOG sensors for eye movements and/or a heart rate monitor e.g., ECG) can also provide additionally useful data for the prediction task. Leveraging EMG sensor data and/or other non-EEG sensors during potential REM window (or time intervals) 208 to validate REM and identify RBD, a structured approach involving one or more techniques may be applied. For example, the data acquired from non-EEG sensors 402 may be extracted for the same time intervals as that of potential REM time intervals. This extracted (or segmented) sensor data may be normalized for consistency and reducing noise that may include removing artifacts and baseline drifts. For these one or more non-EEG sensor data 402, features may be extracted such as amplitude and power (e.g., calculating root mean square (RMS) value), activity index (i.e., count of bursts of activity above a certain threshold), measure of tonic (i.e., sustained low-level muscle activity) and phasic (i.e., short burst or impulsive response) muscle activity. Additionally, statistical measures e.g., entropy, mean, standard deviation, variability can also provide valuable insights for the sensor data over time.

These features from one or more non-EEG sensor(s) 402 may be combined with potential REM time intervals 208 and fed into one or more prediction models trained on labeled data to detect corresponding movements. The segmented data from each sensor corresponding to the REM intervals may be aggregated to form a single observation as input to the prediction model. Alternatively, the segmented data from each of the one or more sensors may be fed individually to one or more prediction models for the detection of muscle tones, abnormal motor activities and/or motion detection during REM sleep. Such prediction models 406 may include machine-learning models configured to capture muscle atonia (i.e., absence of tension in muscles that may indicate a relaxed muscle) and/or body movements during REM sleep leading to reliable detection of RBD. In some instances, the preprocessed data (e.g., normalized and/or denoised) from one or more non-EEG sensors 402 may be combined directly with potential REM time intervals 208 and fed into the one or more prediction models 406. These prediction models 406 may include, but is not limited to, traditional models such as Decision Trees, Random Forest, support vector machine (SVM), or deep learning models such as convolutional neural networks (CNN), recurrent neural network (RNN) or a combination thereof e.g., ensemble methods (i.e., combining predictions from multiple models that takes input from one or more sensors to improve overall performance e.g., combining Random Forest, SVM or deep learning to capture various aspects of one or more sensor data).

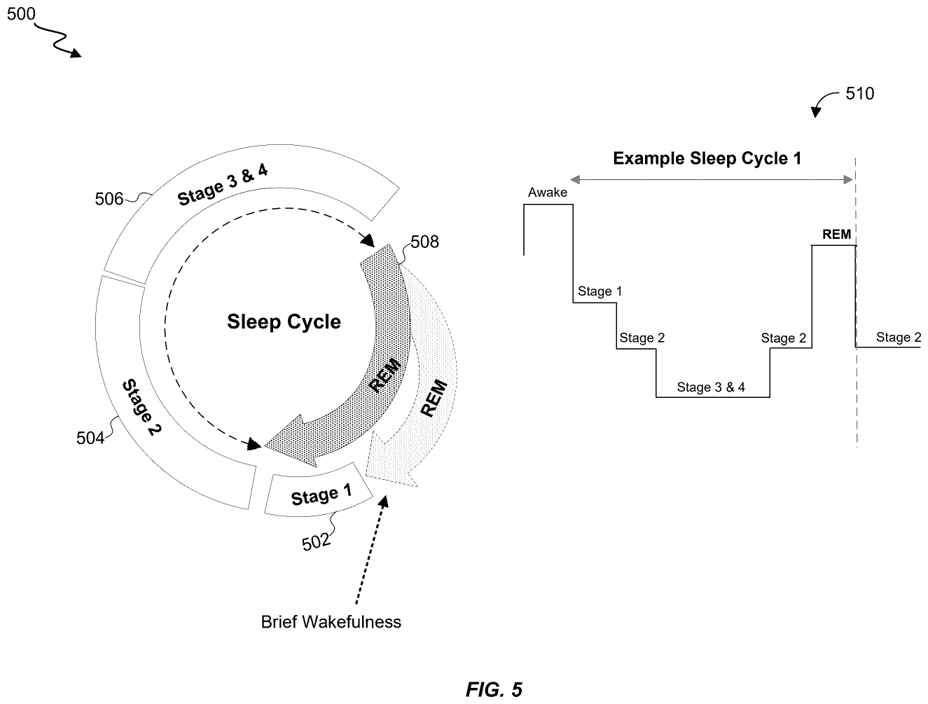

To further validate the correctness of detected REM sleep stages, an analysis of sleep architecture as compared to normal or expected sleep may be performed. FIG. 5 illustrates an example depiction of a normal sleep cycle 500 for a subject. Observing various stages of the sleep cycle 500 and transitions of the sleep stages within the total sleep may also contribute to prediction of RBD. Sleep typically progresses for a significant sleep time (e.g., night sleep of around 8 hours) in sleep cycles of limited time periods (e.g., approximately 90-120 minutes) and repeat throughout the total sleep. Each cycle generally includes a progression from light sleep (i.e., Stage 1 502 that may feature Theta waves) transitioning from wakefulness. This stage generally lasts for a small amount of time of the sleep cycle (e.g., a few minutes) and progresses to Stage 2 504, which is also attributed to lighter sleep but relatively stable than Stage 1 502, and may last more than Stage 1 502 (e.g., for about 10-25 minutes showing sleep spindles and K-complexes). Following Stage 2 504, the sleep may transition into Stage 3 & 4 506 (i.e., deep sleep characterized by high-amplitude, low-frequency Delta waves). This stage may last for a significant time of the initial sleep cycles (e.g., 20-40 minutes) and decrease in duration as the sleep progresses. After the deep sleep (i.e., Stage 3 & 4 504), the subject may often transition back to Stage 2 504 before entering REM sleep 508 that is attributed to e.g., low-amplitude, mixed-frequency EEG patterns similar to wakefulness, REM, muscle atonia and vivid dreaming. The REM window 508 may be relatively short in initial sleep cycles (e.g., around 10-20 minutes) and lengthened in subsequent cycles. Understanding these transitioning of sleep stages may help in validating sleep stages, particularly REM for the detection of neurodegenerative disorders.

Additionally, a sleep pattern analysis may be performed that involves observing the neighboring windows of predicted REM intervals as illustrated in sleep sequence 510 of FIG. 5. REM window may generally follow Stage 2 504 and occasionally follow a short period of Stage 1 502 specifically if the subject wakes up for a brief phase. Alternatively, the subject may return to Stage 2 504 maintaining the continuity of sleep cycle 500 and preparing for the next deep sleep stages (i.e., Stage 3 & Stage 4) 506. To validate REM intervals, it may be helpful to check for these preceding and following stages of REM windows 508.

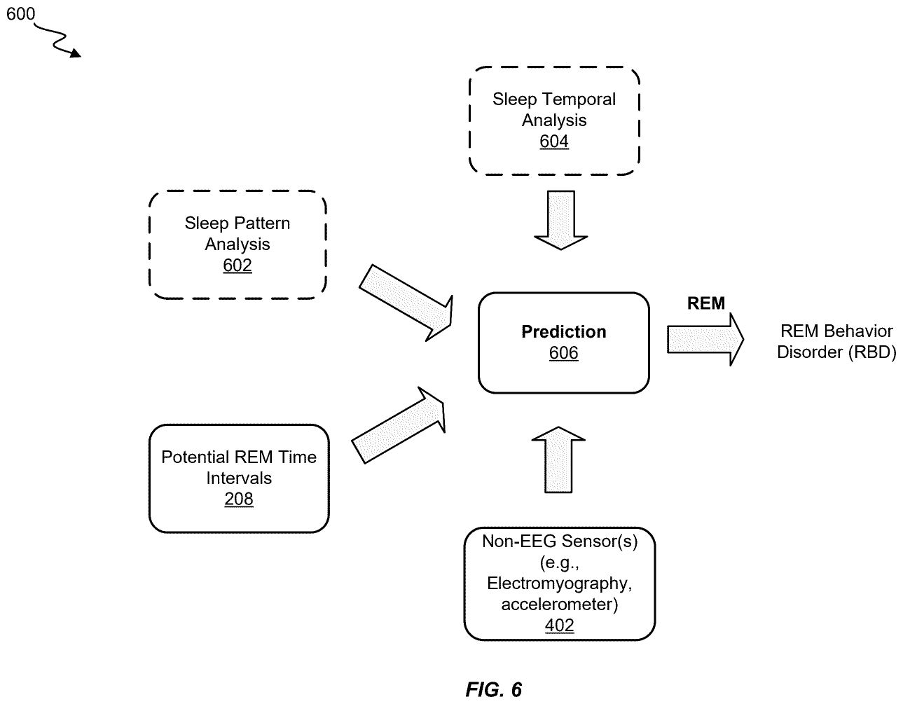

FIG. 6 illustrates an example architecture of predicting REM behavior disorder (RBD) from additional data. The additional data may include sleep pattern analysis 602 and/or sleep temporal analysis 604. In RBD, normal muscle atonia may be absent leading to the subject physically acting out the dreams. Predicting RBD from observing sleep cycles 500 may involve identifying abnormalities during REM sleep and specific patterns that are indicative of disorder. This observation may provide a framework for understanding expected patterns and transitions within the sleep architecture. For example, sleep pattern analysis 602, as discussed before, may be incorporated. The sleep pattern analysis 602 may involve observing neighboring windows for the potential REM time intervals 208. To perform sleep pattern analysis 602 and considering a typical smooth transition between various stages of sleep for a subject (i.e., humans and animals), a smoothing algorithm, a modeling technique, or a statistical analysis may be used to predict—for each potential REM time intervals 208—whether the window corresponds to when the subject was awake or was in REM sleep. For example, if a potential REM window 208 (or a set of consecutive “potential REM” windows) is surrounded (or followed) by “Stage 2” windows, the smoothing algorithm, modeling technique or statistical analysis may be configured to predict that the window (or set of consecutive windows) corresponds to REM sleep. Meanwhile, if a potential REM window 208 (or set of consecutive potential REM windows) is surrounded by “Stage 1” windows, the smoothing algorithm, modeling technique or statistical analysis may be configured to predict that the window corresponds to an awake state. Moreover, if a particular window shows abrupt transitions from one stage to another e.g., from deep sleep directly to REM or vice-versa, it might indicate an incorrectly labeled REM.

For sleep pattern analysis 602, a smoothing technique e.g., a Gaussian filter that may assign a weight to each neighboring window based on its distance from the particular observing window (central point) thus softening abrupt changes, Kalman filter that may predict or forecast the next sleep state based on the current sleep state and observed sleep states data using a weighted average to minimize uncertainty, or a moving average filter that calculates the average label of a series of adjacent windows thus smoothing out the transition between sleep stages to reduce noise. For example, if a particular window is labeled as Stage 1 and surrounded by consecutive windows of Stage 3, it is likely to be a brief arousal or a misclassified interval. The smoothing technique may likely merge the Stage 1 into the surrounding windows i.e. Stage 3.

Alternatively, or in combination, overall sleep temporal analysis 604 may also be observed that may involve a predictable lengthening of REM windows with each successive sleep cycle. For example, given a long sleep time e.g., a night sleep of approximately 8 hours, REM windows 508 tend to lengthen and occur more frequently in the latter half of the total sleep. During the first half of the sleep, more time may be spent in Stage 3 or Stage 4 (deep sleep) 506 and less time in REM sleep 508. Following the first half, in second half, REM windows 508 may lengthen in duration, deep sleep 506 may decrease, and more time may be spent in Stage 2 504 and REM state 508.

Modeling techniques such as recurrent neural network (RNN), long short-term memory network (LSTM), gated recurrent unit (GRU) or Hidden Markov Model (HMM) may be used for sleep pattern analysis 602 and temporal analysis 604. These techniques can model sequential data thereby capturing the temporal context by automatically estimating the relationship between consecutive time intervals. Such modeling techniques can learn temporal structure and consider the probability of transitions between sleep stages. By training the model on labeled sleep data by aggregating time intervals or overlapping windows of time intervals (that may or may not be including EEG data 102 and/or data from one or more non-EEG sensors 402), it can learn statistical relationships and probabilities of transitioning between different sleep states based on the observed sequence and use this information to correct inconsistencies. It should be understood that to perform sleep pattern analysis 602 and sleep temporal analysis 604, one unified model or different models may be trained. Once the model is trained, it may be applied to a new input segmented sleep data to validate sequence of sleep stages. The trained model can look for abnormal patterns in sleep intervals. In RBD, the transitions into or out of REM may occur more frequently or in unusual manner such as frequent interruptions of REM by wakefulness or other stages. Such networks may be trained to classify each interval as REM, awake or non-REM based on the extracted features.

In one aspect, the modeling technique may be equipped with e.g., time-of-night segmentation, which may involve splitting the total sleep into two segments as first half and second half and appending this information with segmented time intervals (e.g., a binary variable (or a bit) can indicate whether each time window belongs to first half or second). This appending may enable more focus on analyzing the distribution of REM sleep throughout the total sleep for performing sleep temporal analysis 604. The prediction results from the modeling technique that may perform sleep pattern analysis 602 and sleep temporal analysis 604 and prediction model 406 that may detect muscle tones during REM window 508 may be combined to obtain a final prediction 606. This combination may be performed by various techniques including simple average, weighted average, majority voting or other similar techniques.

If, for example, a subject has been identified with diagnosis of RBD based on the prediction model 406, one or more actions can be performed. Such predictions may be incorporated into a system that can perform one or more actions related to both clinical interventions and safety measures to improve health of the subject and reduce the risks of injury. These actions may include, but not limited to, generating alerts, notifying the concerned authorities (e.g., medical staff, relatives and/or the subject) for a complete medical evaluation for confirming the diagnosis for a neurodegenerative disease such as Parkinson's, scheduling a consultation with a sleep specialist for thorough neurological examination.

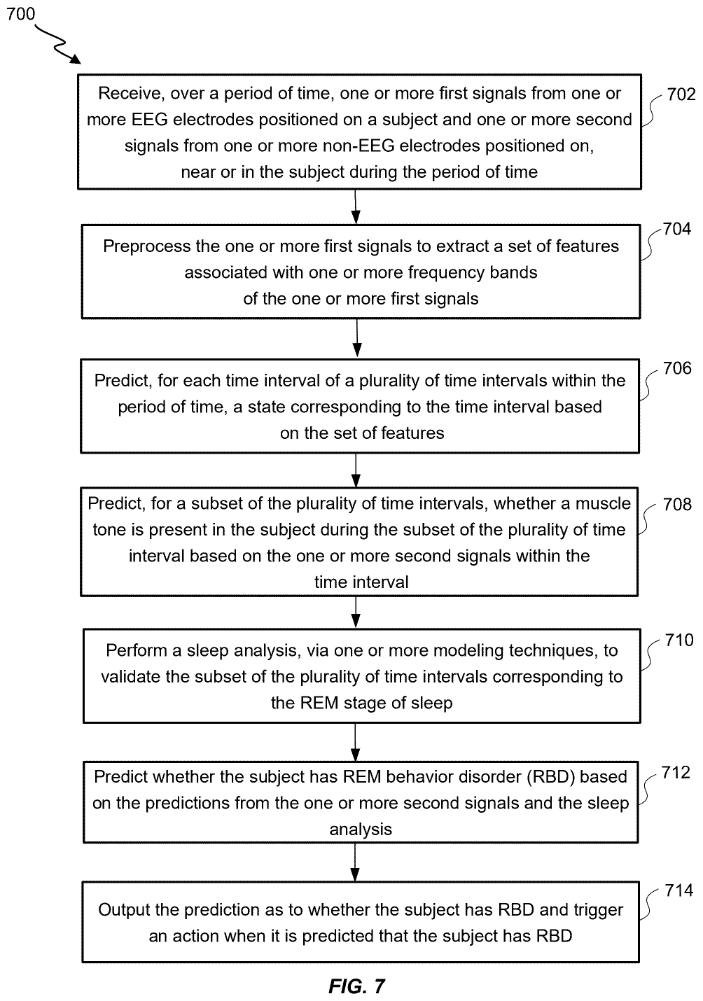

FIG. 7 illustrates an example process flow 700 for determining REM behavior disorder (RBD) by leveraging one or more channels of EEG sensors and further determination of REM behavior disorder (RBD) by leveraging one or more non-EEG sensors that detect muscle tones for the identified REM sleep and performing sleep analysis. The blocks in the process flow 700 are illustrated in a specific order, while the order can be modified, for example, some blocks may be performed before other, and some blocks may be performed simultaneously. The block can be performed by hardware or software or a combination thereof. The process 700 may include receiving one or more first signals from EEG electrodes positioned on a subject over a period of time, at block 702. The EEG data may be received via a single channel (e.g., at least one active electrode, a reference electrode, and potentially a ground electrode) or multiple channels. At block 704, the received data may be preprocessed or adjusted further e.g., for artifact removal, normalization, frequency weighting for selective frequency bands such as Gamma band to extract REM sleep related information. The features and/or derived features may be extracted for the one or more time intervals from these selective spectral bands. These extracted features may be further normalized (e.g., z-scoring) and clustered to determine time intervals that correspond to REM, awake and non-REM (e.g., any one of the sleep Stages 1 to 4), at block 706. Apart from one or more EEG signals, one or more second signals from non-EEG electrodes or sensors (e.g., electromyography (EMG) or accelerometer) may be received. These second signals may be incorporated to check whether a muscle tone is present and/or the subject moved during a subset of predicted (potential) REM sleep.

At block 708, for the predicted subset of the multiple time intervals that correspond to the REM stage, a prediction may be performed as whether the subject has muscle atonia and/or the subject showed any movement during the predicted subset of REM time interval. For this validation, one or more prediction models may be used that takes the data from the one or more non-EEG sensors such as EMG sensors (and/or accelerometers for abnormal motor activity) capturing muscle tone or muscle tension indicative of muscle readiness to perform action, which may be significant for distinguishing between REM, non-REM sleep stages (or wakefulness). The second signal acquired from non-EEG sensors may be extracted for the same time intervals as that of potential REM time intervals. This extracted data from one or more non-EEG sensors may be normalized and/or filtered for noise removal and may be combined with the potential REM time intervals. The one or more prediction models such as a machine-learning models may take this input and capture muscle tones (or muscle activities) or body movements during REM sleep leading to reliable detection of RBD. Alternatively, features may be derived from the one or more non-EEG sensor data and then may be combined with potential REM time interval data and fed into the prediction models.

Additionally, a sleep analysis may be performed, via one or more modeling techniques, to validate the subset of the multiple time intervals corresponding to REM stage of sleep, at block 710. The modeling techniques may be trained to learn a temporal structure comprising different stages of sleep within a given period of time. In some aspects, the one or more modeling techniques may be configured to identify a smooth transition between different stages of sleep by analyzing neighboring time intervals for the subset of the multiple time intervals corresponding to REM stages of sleep. For example, if a specific time interval from the subset of multiple time intervals is surrounded by “Stage 2” windows, the modeling technique may be configured to predict that the specific window (or time interval) corresponds to REM sleep. Moreover, the one or more modeling techniques may also be configured to identify, within the period of time, a gradual increase of a consecutive REM stages of sleep from the subset of the multiple time intervals corresponding to REM stage of sleep.

At block 712, based on the prediction from the non-EEG sensors as to whether subject moved during the predicted subset of multiple time intervals that correspond to REM state and sleep analysis performed by the one or more modeling techniques, it may be predicted whether the subject is diagnosed with RBD. In response to this prediction, at block 714, an output indicating the prediction results of RBD may be shown. If, for example, a subject is diagnosed with RBD, one or more actions can be triggered that may involve preventive measures and medical interventions. For example, these actions may include, but not limited to, generating alerts, notifying the concerned authorities (e.g., medical staff, relatives and/or the subject) for a complete medical evaluation for confirming the diagnosis for a neurodegenerative disease such as Parkinson's, scheduling a consultation with a sleep specialist for thorough neurological examination.

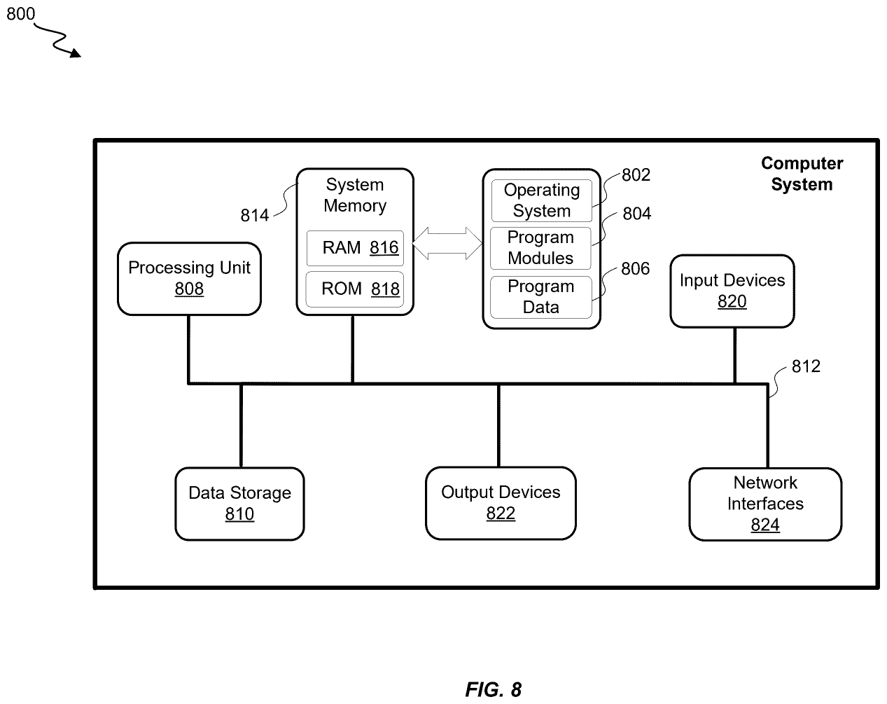

FIG. 8 is an example illustration of a computer system 800 in which various embodiments of the present disclosure may be implemented. For example, the techniques described above performing a determination of rapid eye movement (REM) behavior disorder (RBD) from one or more EEG signals recorded for a subject sleep can be implemented in computer-executable instructions (e.g., organized in program modules 804). The program modules 804 can include the routines, programs, objects, components, and data structures that perform the tasks and implement the data types for implementing the techniques described above. The functionality described herein can be performed, at least in part, by one or more hardware logic components.

To provide additional context for various aspects thereof, FIG. 8 and the following description are intended to provide a brief, general description of the suitable computer system 800 in which the various aspects can be implemented. While the description above is in the general context of computer-executable instructions that can run on one or more computers, those skilled in the art will recognize that a novel implementation also can be realized in combination with other program modules and/or as a combination of hardware and software. Computer system 800 for implementing various aspects includes a processing unit 808 having one or more processors (also referred to as microprocessors), a computer-readable storage medium (where the medium is any physical device or material on which data can be electronically and/or optically stored and retrieved) such as a data storage unit 810 (computer readable storage medium/media also include magnetic disks, optical disks, solid state drives, external memory systems, and flash memory drives), and a system bus 812. The system bus 812 may provide an interface for system components including, but not limited to, system memory 814, to processing unit 808. Such a system bus 812 can be of any of several types of bus structure that can further interconnect to memory bus (with or without controller), and a peripheral bus (e.g., PCI, PCIe, AGP, LPC, etc.), using any of a variety of commercially available bus architectures.

FIG. 8 shows an example configuration of a typical computer that may be other commercially available microprocessors such as single-processor, multi-processor, single-core units, and multi-core units of processing and/or storage circuits. Moreover, those skilled in the art will appreciate that the novel system and methods can be practiced with other computer system configurations, including minicomputers, mainframe computers, as well as personal computers (e.g., desktop, laptop, tablet PC, etc.), hand-held computing devices, microprocessor-based or programmable consumer electronics, and the like, each of which can be cooperatively coupled to one or more associated devices.

In some aspects, the computer system 800 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example. In some instances, system memory 814 can include computer-readable storage (physical storage) medium such as a volatile memory (e.g. random-access memory (RAM) 816) and a non-volatile memory (e.g., (ROM) 818). A basic Input/output system (BIOS) can be stored in the non-volatile memory and includes the basic routines that facilitate the communication of data and signals between components within the computer system 800, such as during startup. The volatile memory also includes a high-speed RAM such as static RAM for caching data.

By way of example, and not limitation, system memory 814 also may also include program modules 804, which may include client applications, Web browsers, mid-tier applications, relational database management systems (RDBMS), etc., program data 806, and an operating system 802. By way of example, operating system 802 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome OS, and the like) and/or mobile operating systems such as iOS, Windows® Phone, Android OS, BlackBerry® OS, and Palm® OS operating systems. All or portions of operating system 802, program modules 804, and/or program data 806 can also be cached in memory such as the volatile memory and/or non-volatile memory, for example (RAM 816 or ROM 818). It is to be appreciated that the disclosed architecture can be implemented with various commercially available operating systems or combinations of operating systems (e.g., virtual machines).

In some other examples, the computer system 800 may have additional features or functionality. For example, the computer system 800 may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer readable instructions, data structures, program modules, or other data.

The system memory 814, and data storage 810 including removable storage, and non-removable storage are all examples of computer storage media. Apart from RAM 816 and ROM 818, computer storage media includes, but is not limited to, EEPROM, flash memory or other memory technology, CD-ROM, digital versatile disks (DVD), or other optical storage, magnetic cassettes, magnetic tape, magnetic disk storage or other magnetic storage devices, or any other non-transmission medium that can be used to store the targeted information and which can be accessed by computer system 800. Moreover, the computer readable media may include computer-executable instructions that, when executed by the processing unit 808, perform various functions and/or operations described herein. In contrast, communication media may embody computer readable instructions, data structures, program modules, or other data in a modulated data signal, such as a carrier wave, or other transmission mechanism.

The computer system 800 may also include one or more input devices 820 such as keyboard, mouse, pen, voice input device, touch input device, etc. One or more output devices 822 such as a display, speakers, printers, etc. may also be included. These devices are well known in the art and are not discussed at length here. The computing device 800 may also include one or more network interfaces 824 to establish communication that may allow computer system 800 to communicate with other system or devices, such as over a network. These networks may include wired networks as well as wireless networks. Here, the computer system 800 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game con soles, programmable consumer electronics, network PCs, minicomputers, mainframe computers, distributed computing environments that include any of the above systems or devices, and/or the like. For example, some or all of the components of computer system 800 may be implemented in a cloud computing environment, such that resources and/or services are made available via a computer network for selective use by the user devices.

Although specific aspects have been described, various modifications, alterations, alternative constructions, and equivalents are possible. Embodiments are not restricted to operation within certain specific data processing environments, but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claim is:

1. A computer-implemented method comprising:
receiving, over a period of time:
one or more first signals from one or more EEG electrodes, wherein at least one of the one or more EEG electrodes was positioned on a subject during the period of time; and
one or more second signals from one or more non-EEG electrodes, wherein at least one or the one or more non-EEG electrodes was positioned on, near or in the subject during the period of time;
preprocessing the one or more first signals to extract a set of features associated with one or more frequency bands of the one or more first signals;
predicting, for each time interval of a plurality of time intervals within the period of time, a state corresponding to the time interval based on the set of features, wherein the state corresponds to any of one or more sleep stages or an awake state, and wherein, for a subset of the plurality of time intervals, the state is a rapid eye movement (REM) stage of sleep;
predicting, for the subset of the plurality of time intervals corresponding to a prediction that the time interval corresponds to the REM stage of sleep, whether a muscle tone is present in the subject during the subset of the plurality of time intervals based on the one or more second signals within the time interval;
performing a sleep analysis, via one or more modeling techniques, to validate the subset of the plurality of time intervals corresponding to the REM stage of sleep, wherein the one or more modeling techniques are trained to learn a temporal structure comprising of the one or more sleep stages within the period of time;
predicting whether the subject has REM behavior disorder based on the prediction from the one or more second signals during the subset of the plurality of time intervals and the sleep analysis;
outputting the prediction as to whether the subject has REM behavior disorder; and
triggering an action when it is predicted that the subject has REM behavior disorder.

2. The computer-implemented method of claim 1, wherein the one or more modeling techniques are configured to identify a smooth transition for the subset of the plurality of time intervals corresponding to the REM stage of sleep by analyzing one or more neighboring time intervals of each time interval of the subset of the plurality of time intervals.

3. The computer-implemented method of claim 1, wherein the one or more modeling techniques are configured to identify, within the period of time, a gradual increase of a consecutive REM stages of sleep from the subset of the plurality of time intervals corresponding to the REM stage of sleep.

4. The computer-implemented method of claim 1, wherein the one or more modeling techniques include Hidden Markov Model (HMM) or recurrent neural network (RNN).

5. The computer-implemented method of claim 1, wherein the one or more first signals include a single-channel EEG data.

6. The computer-implemented method of claim 1, wherein the set of features associated with the one or more frequency bands include one or more of Delta power, Gamma power, standard deviation, maximum amplitude, Gamma power/Delta power, time derivative of Delta, and time derivative of the Gamma power/Delta power.

7. The computer-implemented method of claim 1, further including:
segmenting the one or more first signals and the one or more second signals into the plurality of time intervals.

8. A system comprising:
one or more data processors; and
a non-transitory computer readable storage medium containing instructions which, when executed on the one or more data processors, cause the one or more data processors to perform operations including:
receive, over a period of time:
one or more first signals from one or more EEG electrodes, wherein at least one of the one or more EEG electrodes was positioned on a subject during the period of time; and
one or more second signals from one or more non-EEG electrodes,
wherein at least one or the one or more non-EEG electrodes was positioned on, near or in the subject during the period of time;
preprocess the one or more first signals to extract a set of features associated with one or more frequency bands of the one or more first signals;
predict, for each time interval of a plurality of time intervals within the period of time, a state corresponding to the time interval based on the set of features, wherein the state corresponds to any of one or more sleep stages or an awake state, and wherein, for a subset of the plurality of time intervals, the state is a rapid eye movement (REM) stage of sleep;
predict, for the subset of the plurality of time intervals corresponding to a prediction that the time interval corresponds to the REM stage of sleep, whether a muscle tone is present in the subject during the subset of the plurality of time intervals based on the one or more second signals within the time interval;
perform a sleep analysis, via one or more modeling techniques, to validate the subset of the plurality of time intervals corresponding to the REM stage of sleep, wherein the one or more modeling techniques are trained to learn a temporal structure comprising of the one or more sleep stages within the period of time;
predict whether the subject has REM behavior disorder based on the prediction from the one or more second signals during the subset of the plurality of time intervals and the sleep analysis;
output the prediction as to whether the subject has REM behavior disorder; and
trigger an action when it is predicted that the subject has REM behavior disorder.

9. The system of claim 8, wherein the one or more modeling techniques are configured to identify a smooth transition for the subset of the plurality of time intervals corresponding to the REM stage of sleep by analyzing one or more neighboring time intervals of each time interval of the subset of the plurality of time intervals.

10. The system of claim 8, wherein the one or more modeling techniques are configured to identify, within the period of time, a gradual increase of a consecutive REM stages of sleep from the subset of the plurality of time intervals corresponding to the REM stage of sleep.

11. The system of claim 8, wherein the one or more modeling techniques include Hidden Markov Model (HMM) or recurrent neural network (RNN).

12. The system of claim 8, wherein the one or more first signals include a single-channel EEG data.

13. The system of claim 8, wherein the set of features associated with the one or more frequency bands include one or more of Delta power, Gamma power, standard deviation, maximum amplitude, Gamma power/Delta power, time derivative of Delta, and time derivative of the Gamma power/Delta power.

14. The system of claim 8, further including:
segmenting the one or more first signals and the one or more second signals into the plurality of time intervals.

15. A computer-program product tangibly embodied in a non-transitory machine-readable storage medium, including instructions configured to cause one or more data processors to perform operations including:
receiving, over a period of time:
one or more first signals from one or more EEG electrodes, wherein at least one of the one or more EEG electrodes was positioned on a subject during the period of time; and
one or more second signals from one or more non-EEG electrodes, wherein at least one or the one or more non-EEG electrodes was positioned on, near or in the subject during the period of time;
preprocessing the one or more first signals to extract a set of features associated with one or more frequency bands of the one or more first signals;
predicting, for each time interval of a plurality of time intervals within the period of time, a state corresponding to the time interval based on the set of features, wherein the state corresponds to any of one or more sleep stages or an awake state, and wherein, for a subset of the plurality of time intervals, the state is a rapid eye movement (REM) stage of sleep;
predicting, for the subset of the plurality of time intervals corresponding to a prediction that the time interval corresponds to the REM stage of sleep, whether a muscle tone is present in the subject during the subset of the plurality of time intervals based on the one or more second signals within the time interval;
performing a sleep analysis, via one or more modeling techniques, to validate the subset of the plurality of time intervals corresponding to the REM stage of sleep, wherein the one or more modeling techniques are trained to learn a temporal structure comprising of the one or more sleep stages within the period of time;
predicting whether the subject has REM behavior disorder based on the prediction from the one or more second signals during the subset of the plurality of time intervals and the sleep analysis;
outputting the prediction as to whether the subject has REM behavior disorder; and
triggering an action when it is predicted that the subject has REM behavior disorder.

16. The computer-program product of claim 15, wherein the one or more modeling techniques are configured to:
identify a smooth transition for the subset of the plurality of time intervals corresponding to the REM stage of sleep by analyzing one or more neighboring time intervals of each time interval of the subset of the plurality of time intervals; and
identify, within the period of time, a gradual increase of a consecutive REM stages of sleep from the subset of the plurality of time intervals corresponding to the REM stage of sleep.

17. The computer-program product of claim 15, wherein the one or more modeling techniques include Hidden Markov Model (HMM) or recurrent neural network (RNN).

18. The computer-program product of claim 15, wherein the one or more first signals include a single-channel EEG data.

19. The computer-program product of claim 15, wherein the set of features associated with the one or more frequency bands include one or more of Delta power, Gamma power, standard deviation, maximum amplitude, Gamma power/Delta power, time derivative of Delta, and time derivative of the Gamma power/Delta power.

20. The computer-program product of claim 15, further including:
segmenting the one or more first signals and the one or more second signals into the plurality of time intervals.
